(19) United States
(12) Patent Application Publication
(10) Pub. No.: US 2005/0201891 A1
(43) Pub. Date: Sep. 15, 2005

(54) Recovering energy from carcasses

(76) Inventor: Kevin A. Morris, Zephyrhills, FL (US)

Correspondence Address: DONALD J. ERSLER, S.C., 725 GARVENS AVENUE, BROOKFIELD, WI, 53005, US

(21) Appl. No.: 11/075,538

(22) Filed: Mar. 9, 2005

Related U.S. Application Data
(60) Provisional application No. 60/552,345, filed on Mar. 10, 2004.

(51) Int. Cl. A61L 2/18

(52) U.S. Cl. 422/28; 422/33; 422/36; 422/37; 422/292

(57) ABSTRACT

The present invention involves carcass disposal and particularly to recovering energy from a biological mass while effectively destroying pathogens. Provided is contacting the carcass with a treatment solution and blending the carcass and treatment solution in a ribbon blender under vacuum to form a ground, sterile, dehydrated biological mass. Also provided is a ribbon blender, a vacuum pump in communication with an internal volume of the ribbon blender and a condenser operably coupled with the vacuum pump.

The present application claims priority on provisional patent application Ser. No. 60/552,345, to Morris, filed on Mar. 10, 2004, entitled “Method Of Recovering Energy From Carcasses And Apparatus Therefor”, the contents of which are incorporated by reference herein.

### FIELD OF THE INVENTION

The invention resides in the field of carcass disposal and particularly to methods and an apparatus for recovering energy from a biological mass while effectively destroying pathogens.

### BACKGROUND OF THE INVENTION

Disposal of carcasses has become an increasingly difficult and expensive process due to the imposition of USDA, EPA and EU guidelines requiring disposal means that assure the containment and destruction of biological pathogens, heavy metals and radioactive compounds. The difficulty and expense are greatly compounded in the event of disease outbreaks occurring in domestic and feed animals necessitating large-scale animal destruction and carcass disposal. The known methods of disposing of regulated wastes generated by many universities, health-care, and research facilities face an uncertain future under the ever narrowing scope of environmental laws and regulations. Furthermore, each disposing method is extremely costly, putting an unneeded drain on the already strained resources of universities, health-care organizations, and research facilities.

Previously, carcass disposal means have focused only on effective pathogen destruction without consideration of cost and volume limitations. Thus, the current systems produce an effluent that is typically at least three times the volume of the original carcass. Additionally, the waste generated by the current methods must be either discharged to the sewer, dehydrated, or solidified and taken to the landfill or composted. Sewer use can be prohibitively expensive due to high BOD values of the effluent and dehydration produces uncontrollable offensive odors. For example, U.S. Pat. Nos. 6,437,211 and 6,472,580 disclose methods of disposing of carcasses by submersing the carcass in a highly basic solution and extracting toxic or radioactive substances for separate disposal followed by disposal in septic tanks, sewage systems and local landfills. This results in a significant increase in the total waste disposed and a loss of the energy available in the biological mass.

Thus, a strong need exists for methods of disposing of potentially infectious carcasses and other hazardous materials which is safer, less expensive than currently existing disposal means and results in a diminished volume of waste while preparing the waste for extraction of energy.

### DETAILED DESCRIPTION OF THE INVENTION

The present invention provides a method and apparatus to treat and solidify biological waste especially carcasses. Solidification is the optimal solution, but current technology requires a separate mechanical unit to achieve this goal. This results in increased operational costs and decreased efficiency. The closed system of the present invention modifies existing technologies to convert carcasses into usable materials in cost-effective methods.

The present invention uses equipment, similar to that which is used to produce bonemeal, and additional methods to convert carcasses into usable energy and/or useful materials. The products of the process can be used for safe composts and fertilizers guaranteeing that all pathogens are destroyed, or be used for the production of Bio-Diesel. The process destroys all known pathogens, including prions, according to USDA, EPA, and EU accepted guidelines.

In methods of the present invention, carcasses are loaded into the unit with mechanical ribbon shredders, the lid is secured, desired chemicals added and the unit heated to 350° F. Moisture and gases generated during the process are extracted by the vacuum system. The volume left is 10% to 30% of the original weight, depending on the level of moisture desired, and is discharged into a transport container. The closed system prevents any odors emitted and produces a product that can be profitably and safely used as compost, fertilizer, or Bio-Diesel. This system can be scaled-up to as much as a 35,000 lb handling capacity, and multiple mobile units can be used to meet any potential emergency needs.

In the method of the present invention, the biological mass is loaded into a ribbon blender to which a treatment liquid is added. The biological waste is macerated in the ribbon blender in the presence of the treatment liquid while a vacuum is applied to the blender to remove moisture and gasses from the biological mass. The grinding may take place at an elevated temperature to enhance dehydration of the biological mass. The biological mass is macerated and dehydrated until either partially or completely dried. After the biological mass has been dehydrated to a desired dryness, the biological mass is removed from the blender for use as compost or further processing into Bio-Diesel.

The biological mass may be any biological, animal medical, or research waste including, but not limited to, blood, bodily fluids, carcasses, microorganisms, biological samples, fermentation waste and the like. Typically, the biological mass is carcass material. The biological mass may be loaded into the ribbon blender by any appropriate means such as overhead cranes, pedestal cranes, hoists, gutbucket rotators, augurs, carts and the like so long as care is taken not to contaminate the personnel and/or the area outside of the ribbon blender with any infectious or toxic agents that may be present in the biological mass.

The ribbon blender may be any commercial ribbon blender that can be fitted with a vacuum attachment and is sufficiently water tight to hold the treatment liquid. A condenser is then attached to the vacuum such that liquids and gasses removed from the biological mass during processing in the ribbon blender can be captured and condensed to a liquid form. These liquids can be reused in the processing of additional biological masses or disposed of without further processing. The ribbon blender may be of any size that can accommodate the biological mass to be processed. Commercially available ribbon blenders have capacity ranging from 10 pounds to over 35,000 pounds. The ribbon blender with vacuum and condenser attachments may also be mounted to allow mobile transport to areas where the disposal of biological masses, particularly carcasses, is needed thereby avoiding or minimizing the transport and handling of potentially infectious or toxic biological masses. Preferably, the ribbon blender is a horizontal ribbon blender fitted with a vacuum such as those available from Ross.

The liquid used in liquid treatment may be any liquid that will impart the desired properties to the macerated biological mass during the treatment in the blender. For example, the treatment of the present invention may include sterilization and dehydration of the biological mass for safe disposal following treatment. In this instance, the treatment solution may be a sterilization solution such as caustic, sodium hypochlorite, germicide and/or glutaraldehyde. Alternatively, the treatment goal may be the production of a dry waste for compost or further processing to Bio-Diesel. In this instance, the treatment solution may be water or petroleum products compatible with the desired end product.

The vacuum applied during maceration of the biological mass in the presence of the liquid treatment need not be very strong. Even a slight vacuum will accomplish the desired removal of moisture and gases generated in the blender vessel without pulling solids from the contents of the blender. These gases can be separated and recovered in a condenser attached to the vacuum pump. The liquid removed by the vacuum as well as the liquid captured as gas and condensed can be pooled and used in subsequent runs of the ribbon blender or disposed of without further use. Removing and capturing the liquid and gases present in the blender greatly reduce the volume of the biological mass creating a final product that is partially or completely dry and much easier to handle than the double or triple volume of liquid waste created by prior art processes while leaving only the re-useable fuels from the treated biological mass.

The temperature of the blender and its contents are elevated during the maceration of the biological mass in the presence of the treatment liquid. The temperature may range from ambient temperature to about 600° F. Preferably, the temperature is raised to between about 350° F. to about 400° F. during the maceration. The temperature is raised slowly and may be lowered before the anticipated end of the maceration to cool the contents of the blender prior to stopping the maceration and removal of the contents of the blender. Alternatively, the blender and its contents may be heated throughout the maceration and the contents of the blender allowed to cool only after the maceration has ended. The biological mass may also be heated partially or completely in the blender with or without maceration prior to contact with the treatment liquid. In one embodiment, the biological mass is macerated and heated to not higher than about 200° F. prior to addition of the treatment liquid to the ribbon blender. Thereafter, maceration and heating can continue at higher temperatures under vacuum.

The maceration, heating and vacuum are continued until the biological mass has achieved the dryness desired. The desired dryness may range from only slightly dry to almost completely or completely dry. The level of dryness desired in the product is determined by the intended use of the product after treatment. For example, if the treated biological mass is to be used as a source of energy for a composting pile, the treatment time and temperature would be adjusted to leave moisture in the product. Alternatively, if the product is not to be recovered but is intended for landfill, the product is treated until as dry as possible to reduce the volume and ease in handling of the product and meet landfill requirements for non-aggressive and non-flowing waste. This product is sterile, dry and free of disease. Typically, the treatment is continued for between about 3 and about 12 hours. Preferably, the treatment is continued between about 6 and about 9 hours.

The product may be removed from the blender following maceration. The blender contents can be removed through the top of the blender or by center or end discharge doors. The product removed may then be further processed into dry solid fuels, fertilizers, composts, Bio-Diesel and the like as desired.

1. A method of treating an animal carcass comprising:
contacting the carcass with a treatment solution; and,
blending the carcass and treatment solution in a ribbon blender under vacuum to form a ground, sterile, dehydrated biological mass.

2. The method of claim 1, wherein the blending under vacuum continues until the biological mass is completely dehydrated.

3. The method of claim 1, wherein the blending under vacuum continues until the biological mass is partially-dehydrated.

4. The method of claim 1, wherein the treatment solution is selected from the group consisting of an acidic solution, a basic solution, a sterilizing solution, and water.

5. The method of claim 4, wherein the treatment solution is a basic solution selected from the group consisting of sodium hydroxide, potassium hydroxide, and calcium hydroxide.

6. The method of claim 4, wherein the treatment solution is a sterilization solution selected from the group consisting of sodium hypochlorite, germicides, and glutaraldehydes.

7. The method of claim 1, wherein the blending step is conducted at a temperature between ambient temperature and about 600° F.

8. The method of claim 7, wherein the blending step is conducted at a temperature between about 350° F. and about 400° F.

9. The method of claim 1, wherein the blending step is conducted for a time period between about 3 hours and about 12 hours.

10. The method of claim 1, wherein the blending step is conducted for a time period between about 6 hours and about 9 hours.

11. The method of claim 1, comprising the additional step of macerating the carcass in a ribbon blender while heating to a temperature of at least about 200° F. prior to the contacting step.

12. The method of claim 1, wherein the carcass is completely immersed in the treatment solution.

13. The method of claim 1, wherein the carcass is partially immersed in the treatment solution.

14. A device for treating biological waste comprising:
a ribbon blender;
a vacuum pump in communication with an internal volume of the ribbon blender; and
a condenser operably coupled with the vacuum pump.

15. The device of claim 14, wherein the vacuum pump is connected to the ribbon blender such that vacuum pump is able to modify the internal pressure of the ribbon blender.

16. The device of claim 14, wherein the ribbon blender is a sealable blender that is fitted with a vacuum attachment.

17. The device of claim 14, wherein the ribbon blender is sufficiently sealed to prevent treatment solution and vapors from leaking.

18. The device of claim 14, wherein a vacuum is applied to the blender through the vacuum pump to remove moisture and gasses from the biological mass

19. The device of claim 14, wherein the condenser is attached to the vacuum pump separating and recovering the gases generated in the blender.
